(19) United States
(12) Patent Application Publication
(10) Pub. No.: US 2026/0116908 A1
(43) Pub. Date: Apr. 30, 2026

(54) TYLOSIN DERIVATIVE AND PREPARATION METHOD AND APPLICATION THEREOF

(71) Applicant: Institute of Feed Research, Chinese Academy of Agricultural Science, Beijing (CN)

(72) Inventors: Xiubo LI, Beijing (CN); Yiming Liu, Beijing (CN); Fei XU, Beijing (CN); Xiaojie Chen, Beijing (CN)

(73) Assignee: Institute of Feed Research, Chinese Academy of Agricultural Science, Beijing (CN)

(21) Appl. No.: 19/469,382

(22) PCT Filed: May 31, 2024
(86) PCT No.: PCT/CN2024/096578

§ 371 Date: Sep. 26, 2025

(30) Foreign Application Priority Data
Nov. 30, 2023 (CN) 202311620626.8

(51) Int. Cl. C07H 17/08; A61K 31/7056; A61P 31/04; C07H 1/00

(52) U.S. Cl. CPC C07H 17/08; A61K 31/7056; A61P 31/04; C07H 1/00

(57) ABSTRACT

A tylosin derivative and a preparation method and application thereof are provided. The tylosin derivative has a structure represented by in Formula 1. A veterinary pharmaceutical composition and a pharmaceutical preparation containing the tylosin derivative, and use of them in treating or preventing bacterial infection diseases in animals are also provided. Moreover, a feed additive including the tylosin derivative is provided.

### TECHNICAL FIELD

The present invention belongs to the technical field of the synthesis of heterocyclic compounds, veterinary drugs for livestock and poultry and feed additives, and specifically relates to a tylosin derivative and a preparation method and application thereof.

### BACKGROUND ART

Tylosin is an important antibiotic for animals, which has a 16-membered macrolide structure and was first extracted from the culture medium of Streptomyces fradiae. This product has unique therapeutic effects on diseases such as Mycoplasma gallisepticum infection and swine epidemic pneumonia, and can be used as a feed additive to significantly promote the growth of livestock and poultry. In order to further expand the antibacterial spectrum and application of tylosin and to develop new antibiotic, researchers have made various modifications to the structure of tylosin and obtained a variety of tylosin derivatives with strong antibacterial activity and low toxicity and side effects. For example, 10,11,12,13-tetrahydrodesmycosin derivative, 9-oximidotylosin derivatives, Tildipirosin, Tilmicosin, Tylvalosin, and so on.

### CONTENT OF THE INVENTION

The object of the present invention is to provide a new derivative of tylosin and a preparation method and application thereof, which can be used to treat or prevent bacterial infection and to provide more selectivity for treatment with tylosin derivatives.

To achieve the above object, the present invention provides the following technical solutions:

In a first aspect, the present invention provides a tylosin derivative having a structure represented by in Formula 1:

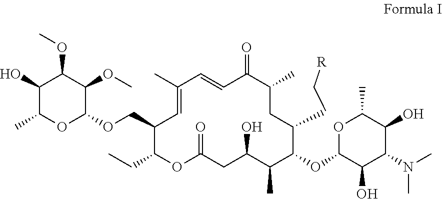

wherein R is selected from the group consisting of 2-hydroxyethylamino, 3-hydroxypropylamino, 4-hydroxybutylamino, 5-hydroxypentylamino, di(2-hydroxyethylamino), di(3-hydroxypropylamino), di(4-hydroxybutylamino), (R)-2-hydroxymethyltetrahydropyrrolyl, (S)-2-hydroxymethyltetrahydropyrrolyl, (R)-3-hydroxymethyltetrahydropyrrolyl, (S)-3-hydroxymethyltetrahydropyrrolyl, (R)-3-hydroxymethylpiperidyl, (S)-3-hydroxymethylpiperidyl, 4-hydroxypiperidyl, 4-hydroxymethylpiperidyl, and 4-hydroxyethylpiperidyl.

By way of example, the tylosin derivative is selected from one of the group consisting of: 20-(2-hydroxyethylamino)desmycosin, 20-(3-hydroxypropylamino)desmycosin, 20-(4-hydroxybutylamino)desmycosin, 20-(5-hydroxypentylamino)desmycosin, 20-(bis(2-hydroxyethylamino))desmycosin, 20-(bis(3-hydroxypropylamino)) desmycosin, 20-(bis(4-hydroxybutylamino))desmycosin, 20-((R)-2-hydroxymethyltetrahydropyrrolyl)desmycosin, 20-((S)-2-hydroxymethyltetrahydropyrrolyl)desmycosin, 20-((R)-3-hydroxymethyltetrahydropyrrolyl)desmycosin, 20-((S)-3-hydroxymethyltetrahydropyrrolyl)desmycosin, 20-((R)-3-hydroxymethylpiperidyl)desmycosin, 20-((S)-3-hydroxymethylpiperidyl)desmycosin, and 20-(4-hydroxypiperidyl)desmycosin.

Further, the tylosin derivative is selected from the compounds having a structure represented by in the following Formula Ia or Ib:

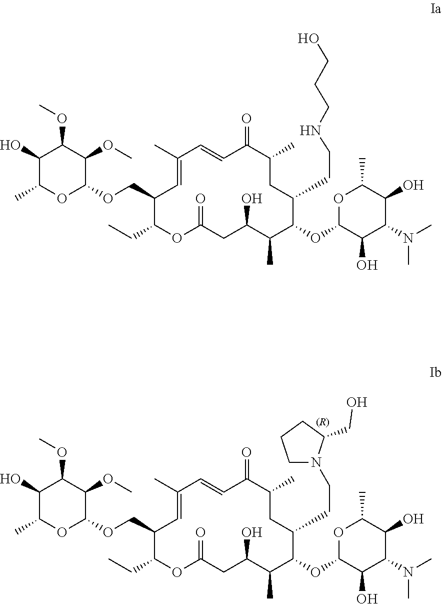

The present invention also provides pharmaceutically acceptable salts of the tylosin derivative as described above. The salts are obtained by reacting the tylosin derivative with an acid. The acid is hydrochloric acid, phosphoric acid, tartaric acid, salicylic acid, methanesulfonic acid, lactic acid, malic acid, formic acid, acetic acid, propionic acid, fumaric acid, citric acid, oxalic acid, maleic acid, succinic acid, benzoic acid, ethanedisulfonic acid, and so on.

In a second aspect, the present invention further provides a preparation method of the tylosin derivative as described above, including the following steps of:
S1. reacting tylosin A with an amino alcohol;
S2. adding a reducing agent or an acid to the system obtained by the reaction in step S1, and reacting to obtain a macrolide compound intermediate; and
S3. hydrolyzing the macrolide compound intermediate under acidic conditions to obtain the tylosin derivative.

Further, the preparation method includes a synthesis method 1 and a synthesis method 2:

The synthesis method 1 includes the following steps of:
(1) subjecting the tylosin A and the amino alcohol to a condensation reaction in a polar solvent, and then adding a reducing agent therein to perform a reduction reaction to obtain a secondary amine-modified macrolide compound intermediate; and
(2) hydrolyzing the secondary amine-modified macrolide compound intermediate under acidic conditions to obtain the tylosin derivative.

In step (1), the amino alcohol is 2-aminoethanol or 3-aminopropanol.

In step (1), the molar ratio of the amino alcohol to the tylosin A is 2:1 to 5:1, preferably from 3:1 to 3.5:1.

In step (1), the polar solvent is selected from one or more of the group consisting of methanol, ethanol, propanol, isopropanol, n-butanol and ethylene glycol.

In step (1), the conditions for the condensation reaction include: a temperature of room temperature, and a time period of 12-13 h.

In step (1), the reducing agent is selected from one or more of the group consisting of sodium borohydride, sodium triacetoxyborohydride and LiAlH₄.

In step (1), the molar ratio of the reducing agent to the tylosin A is from 1:4 to 1:1, preferably from 2:1 to 2.5:1. Before the addition of the reducing agent, TLC is performed to monitor the reaction, ensuring that the raw materials are completely converted into imine.

In step (1), the conditions for the reduction reaction include: a temperature of room temperature, and a time period of 2-6 h, preferably 2 h.

In step (2), the acid is formic acid.

In step (2), the hydrolyzing conditions include: a temperature of room temperature, a time period of 1-6 h, and a concentration of an aqueous solution of the acid is from 0.1 to 10 M, preferably 0.2 M.

Further, the synthesis method 1 also includes post-treatment steps. The post-treatment is carried out in accordance with the following operations: adding an aqueous solution of a base into the reaction system to quench the reaction, and then concentrating it under reduced pressure to remove the alcohol solvent; extracting the remaining aqueous solution with an organic solvent, washing the combined organic phase with a saturated salt solution, drying it over anhydrous sodium sulfate, and concentrating it under reduced pressure; wherein the base is selected from one or more of the group consisting of potassium carbonate, sodium carbonate, potassium hydroxide or sodium hydroxide; and the organic solvent is selected from one or more of the group consisting of dichloromethane, ethyl acetate or diethyl ether.

By way of example, the synthetic route of the synthesis method 1 as described above is as follows:

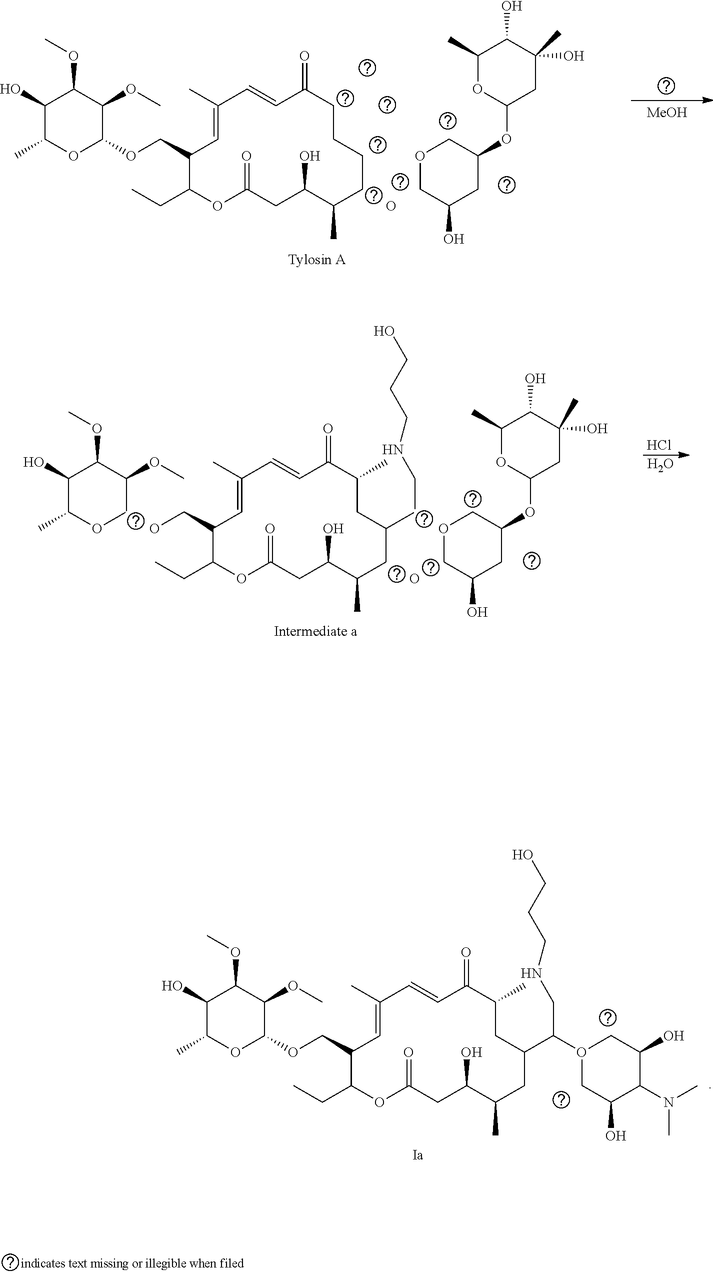

The synthesis method 2 includes the following steps of:
step A: mixing the tylosin A with the amino alcohol in a non-polar solvent, heating and then adding an acid for reacting to obtain a tertiary amine-modified macrolide compound intermediate; and
step B: hydrolyzing the tertiary amine-modified macrolide compound intermediate under acidic conditions to obtain the tylosin derivative.

In step A, the amino alcohol is 2-aminoethanol, 3-aminopropanol, (R)-prolinol, (S)-prolinol, 4-hydroxypiperidine, or 4-hydroxymethylpiperidine.

In step A, the molar ratio of the amino alcohol to the tylosin A is from 2:1 to 5:1, preferably from 2.5:1 to 3.5:1.

In step A, the non-polar solvent is selected from one or more of the group consisting of ethylene glycol dimethyl ether, benzene and toluene.

In step A, the acid is formic acid.

In step A, the acid is added when the temperature of the reaction system reaches 75-85° C., preferably 80° C.

In step A, the molar ratio of the acid to the tylosin A is from 3:1 to 6:1, preferably from 5:1 to 6:1.

In step A, the reaction conditions include: a temperature of 78-80° C. and a time period of 2-6 h.

In step B, the acid is selected from one or more of the group consisting of formic acid, acetic acid, hydrochloric acid and sulfuric acid.

In step B, the hydrolyzing conditions include: a temperature of room temperature, a time period of 1-6 h, and a concentration of an acid aqueous solution is from 0.1 to 10 M, preferably 0.2 M.

Further, the synthesis method 2 also includes post-treatment steps; and the post-treatment is carried out in accordance with the following operations: adding distilled water to the reaction system, adjusting the pH of the aqueous phase obtained after the separation of liquid to 9-11 with a base, extracting the aqueous solution with an organic solvent, washing the combined organic phase with a saturated salt solution, drying it over anhydrous sodium sulfate, and concentrating it under reduced pressure; wherein the base is selected from one or more of the group consisting of potassium carbonate, sodium carbonate, potassium hydroxide or sodium hydroxide; and the organic solvent is selected from one or more of the group consisting of dichloromethane, ethyl acetate or diethyl ether.

Further, the preparation method of the macrolide compound provided by the present invention also includes a purification step: adding the obtained crude product into a silica gel chromatography column, selecting two organic solvents to prepare eluents with different polarities, and using gradient elution to remove impurities from the crude product, thereby obtaining a pure product of macrolide compound; wherein the eluents may be selected from any two of the group consisting of diethyl ether, ethyl acetate, methanol, isopropanol, acetone or dichloromethane.

By way of example, the synthetic route of the synthesis method 2 as described above is as follows:

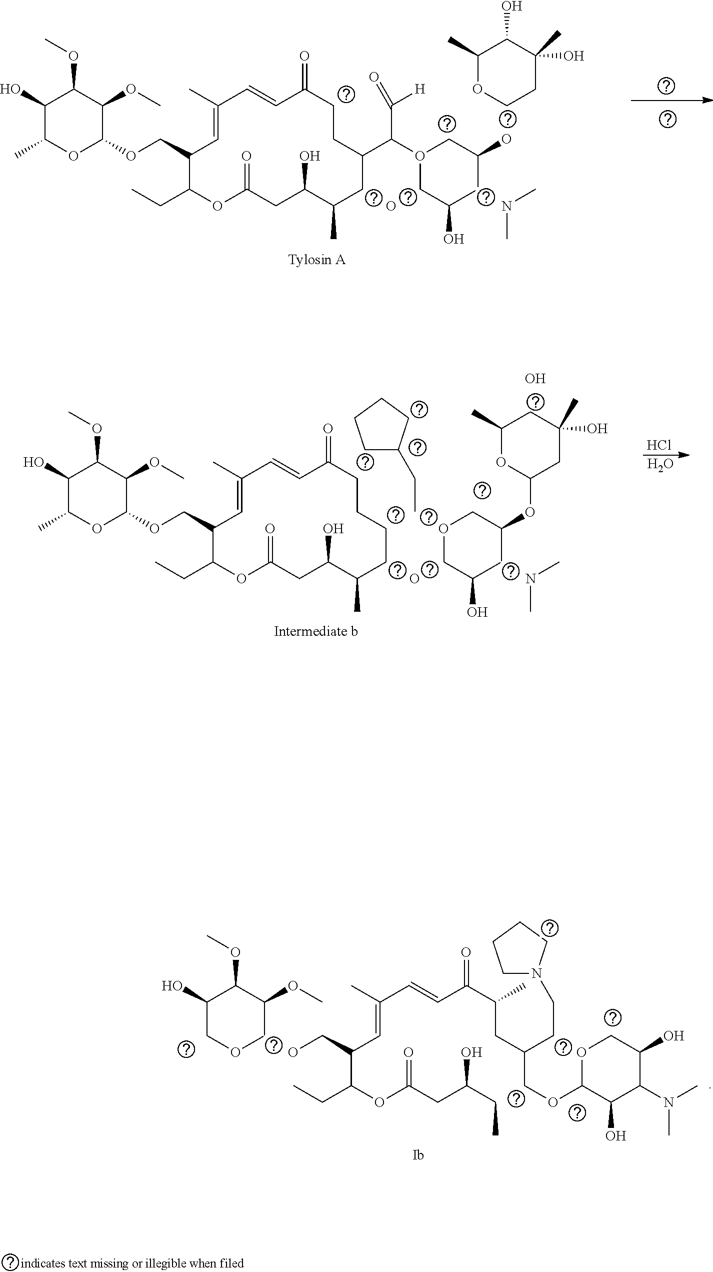

In a third aspect, the present invention further provides a pharmaceutical composition or veterinary pharmaceutical composition, which includes the tylosin derivative having the structure represented by in the Formula I as described above.

In a fourth aspect, the present invention further provides a pharmaceutical preparation, which includes the tylosin derivative having the structure represented by in the Formula I as described above.

The pharmaceutical preparation has a dosage form of powder, tablet, premix, soluble powder, injection.

In a fifth aspect, the present invention further provides use of the above tylosin derivative, veterinary pharmaceutical composition and pharmaceutical preparation in the preparation of a medicament for treating or preventing bacterial infection diseases in animals. By way of example, the anti-pathogen infection pharmaceuticals are products for clinical use for livestock and poultry veterinarians.

In the application, the bacteria include: Staphylococcus aureus, Streptococcus agalactiae, Streptococcus pneumoniae, β-hemolytic Streptococcus, Escherichia coli, Haemophilus influenzae, Moraxella meningitidis, Pasteurella, Actinobacillus, Bordetella, Mycoplasma bovis, Actinobacillus pleuropneumoniae, Salmonella, Erysipelothrix rhusiopathiae, and Bacillus anthracis, and so on.

In a sixth aspect, the present invention further provides a feed additive, which includes the tylosin derivative having the structure represented by in the Formula I as described above.

### Specific Implementations

The present invention will be further set forth below combined with specific examples. However, the present invention is not limited to the following examples.

Unless otherwise specified, the experimental methods used in the following examples are conventional methods.

Unless otherwise specified, the reagents, materials, instruments and so on used in the following examples are commercially available.

### Example 1

The steps were as follows:

(1) Tylosin A (3.00 g, 3.27 mmol) was dissolved in methanol (18 mL), 3-amino-1-propanol (0.74 g, 9.85 mmol) was added therein at room temperature, and it was stirred for reacting. When the raw material Tylosin A could not be detected by TLC, the reaction was stopped to obtain a solution of an imine derivative of Tylosin A.

(2) Sodium triacetoxyborohydride (1.39 g, 6.56 mmol) was added at room temperature, the reaction system was stirred for 2 h, and the reaction was stopped. An aqueous NaOH solution (3 mL, 1 M) was added to quench the reaction, and MeOH was removed by concentration using a rotary evaporator. The residue was extracted with dichloromethane (10 mL×3). The organic phases were combined, washed with a saturated salt solution (10 mL), and dried over anhydrous sodium sulfate. Concentration was carried out using a rotary evaporator, and purification was performed by silica gel column chromatography (dichloromethane/methanol=8:1) to obtain a secondary amine-modified macrolide compound intermediate (1.40 g).

(3) 10 mL of an aqueous hydrochloric acid solution (0.2 M) was prepared and added into a 50 mL pear-shaped flask. Then the secondary amine-modified macrolide compound intermediate (0.50 g, 0.82 mmol) obtained in step (2) was added therein and the reaction system was stirred at room temperature for 2 h. After the completion of the reaction was detected by TLC, the reaction was stopped. The pH of the reaction solution was adjusted to 10 with an aqueous NaOH solution (1 M), and then the reaction solution was extracted with dichloromethane (20 mL×3). The organic phases were combined, washed with a saturated salt solution (10 mL), and dried over anhydrous sodium sulfate. Concentration was carried out with a rotary evaporator and purification was performed with a silica gel chromatography column (dichloromethane/methanol=8:1) to obtain a white solid, tylosin derivative Ia (0.43 g, a yield of 63%).

¹H NMR (500 MHZ, CDCl₃) δ 7.35 (d, J=15.0 Hz, 1H), 6.30 (d, J=15.3 Hz, 1H), 5.92 (J=10.2 Hz, 1H), 4.95-4.93 (m, 1H), 4.57 (d, J=7.6 Hz, 1H), 4.29-4.27 (m, 1H), 4.01-3.99 (m, 1H), 3.79-3.74 (m, 5H), 3.61-3.60 (m, 4H), 3.55 (t, J=7.9 Hz, 3H), 3.48-3.47 (m, 4H), 3.29-3.27 (m, 2H), 3.18 (d, J=9.0 Hz, 1H), 3.13-3.09 (m, 1H), 3.02-2.95 (m, 3H), 2.81-2.77 (m, 2H), 2.71-2.63 (m, 3H), 2.51-2.47 (m, 8H), 1.98-1.84 (m, 3H), 1.78-1.75 (m, 5H), 1.65-1.61 (m, 3H), 1.54-1.52 (m, 2H), 1.33-1.19 (m, 12H), 1.03 (d, J=7.0 Hz, 3H), 0.93 (t, J=7.4 Hz, 3H).

¹³C NMR (126 MHz, CDCl₃) δ 203.90, 173.51, 148.05, 142.83, 134.56, 117.96, 104.00, 101.01, 81.70, 79.84, 79.42, 74.74, 73.12, 72.68, 70.85, 70.74, 70.34, 70.20, 69.03, 66.52, 62.31, 61.64, 59.51, 53.43, 47.60, 46.25, 44.97, 41.64, 41.28, 39.41, 33.50, 32.31, 31.03, 29.54, 26.80, 25.19, 17.68, 17.57, 12.80, 9.55, 9.24.

TLC R_{f}=0.1 (dichloromethane/methanol=8:1)

HRMS (ESI, m/z): [M+H]⁺ calcd for C₄₂H₇₅N₂O₁₄, 831.52128. found 831.52167.

### Example 2

The steps were as follows:

(1) Tylosin A (0.50 g, 0.55 mmol) was dissolved in toluene (6 mL), and (R)-prolinol (0.17 g, 1.68 mmol) was added therein, and it was stirred for dissolving.

(2) The reaction solution was then heated to 80° C., and formic acid (0.14 g, 3.04 mmol) was added therein. The reaction was continued at 80° C. When the raw material Tylosin A could not be detected by TLC, the reaction was stopped. Distilled water (5 mL) was added to quench the reaction and the separation was performed. The aqueous phase was adjusted to pH 10 with an aqueous sodium hydroxide solution (5 M) and extracted with dichloromethane (15 mL×3). The organic phases were combined and dried over anhydrous sodium sulfate. The concentration was carried out using a rotary evaporator and purification was performed by silica gel column chromatography (dichloromethane/methanol=8:1) to obtain a tertiary amine-modified macrolide compound intermediate (0.32 g, a yield of 58%).

(3) 14 mL of an aqueous hydrochloric acid solution (0.2 M) was prepared, added into a 50 mL pear-shaped flask, and then the tertiary amine-modified macrolide compound intermediate (0.70 g, 0.70 mmol) was added therein, and the reaction was stirred at room temperature for 2 h. After the completion of the reaction was detected by TLC, the reaction was stopped. The pH of the reaction solution was adjusted to 10 with an aqueous NaOH solution (1 M), and then the reaction solution was extracted with dichloromethane (30 mL×3). The organic phases were combined, washed with a saturated salt solution (15 mL), and dried over anhydrous sodium sulfate. The organic phase was concentrated with a rotary evaporator and purified with silica gel column chromatography (dichloromethane/methanol=8:1) to obtain a white solid, tylosin derivative Ib (0.58 g, a yield of 97%).

¹H NMR (500 MHZ, CDCl₃) δ 7.35 (d, J=15.3 Hz, 1H), 6.29 (d, J=15.1 Hz, 1H), 5.94 (s, 1H), 4.96 (d, J=9.2 Hz, 1H), 4.59-4.56 (m, 1H), 4.33-4.29 (m, 1H), 4.01-3.98 (m, 1H), 3.83-3.81 (m, 1H), 3.76-3.73 (m, 1H), 3.62-3.57 (m, 6H), 3.47-3.46 (m, 2H), 3.34-3.30 (m, 1H), 3.18-3.13 (m, 2H), 3.04-2.94 (m, 3H), 2.73-2.63 (m, 3H), 2.58-2.51 (m, 13H), 2.39-2.38 (m, 1H), 2.29 (s, 1H), 2.00-1.85 (m, 4H), 1.81-1.72 (m, 6H), 1.63-1.57 (m, 4H), 1.31-1.25 (m, 6H), 1.20-1.19 (m, 3H), 1.06-1.03 (m, 9H), 0.92 (t, J=7.2 Hz, 3H).

¹³C NMR (126 MHZ, CDCl₃) δ 204.10, 173.60, 148.01, 143.05, 134.37, 117.96, 104.21, 100.98, 82.18, 81.64, 79.86, 77.38, 77.13, 76.87, 74.87, 73.22, 72.65, 70.71, 70.29, 70.25, 69.06, 66.49, 65.06, 63.12, 61.59, 59.43, 55.05, 54.66, 45.95, 45.08, 41.61, 39.35, 34.96, 34.20, 27.51, 26.73, 25.17, 23.43, 17.81, 17.65, 12.70, 11.19, 9.56.

TLC R_{f}=0.1 (dichloromethane/methanol=8:1)

HRMS (ESI, m/z): [M+H]⁺ calcd for C₄₄H₇₇N₂O₁₄, 857.53693. found 857. 53705.

Test Example 1 Determination of the antibacterial activity of the compounds of the present invention

The antibacterial activity of the tylosin derivative Ia and the antibacterial activity of the tylosin derivative Ib of the present invention were determined by using the broth microdilution method with tylosin as a positive control.

The specific test method is as follows:

A broth culture medium was added into a 96-well plate, a prepared compound solution was diluted in a trace two-fold decreasing concentration, and then was inoculated with an appropriate amount of bacterial solution. After incubation for 24 hours, the minimum inhibitory concentration of the compound was observed.

The culture medium used in the experiment was CAMHB broth and CAMHB+5% defibrillated sheep blood broth.

The preserved bacteria strain was inoculated into a serum plate medium and cultured at 37° C. for 16-18 hours. An appropriate amount of bacteria after the completion of subculture and saline were placed into a turbidimetric tube and calibrated to the McFarland standard with a McFarland turbidimeter. The bacterial suspension was diluted 10 times with saline to prepare test bacterial solutions of certain concentrations (5×10⁵ to 5×10⁶ cfu/mL) for later use.

Tylosin and the compounds obtained in the examples were dissolved in methanol to the desired concentration of each compound (1.0 mg/mL), stored in sterilized brown vials, stoppered, and sealed for later use. The working concentration range against Gram-negative bacteria was 0.25 μg/mL to 128 μg/mL; and the working concentration range against Gram-positive bacteria was 0.098 g/mL to 50 μg/mL.

The 96-well plate micro double dilution method was used. A broth culture medium was added into the 96-well plate, and the prepared compound solution was diluted in two-fold decreasing concentration, so that the concentrations of the compound solutions from the first well to the tenth well showed a two-fold decreasing tendency, and neither the eleventh well nor the twelfth well was added with the compound solution. Finally, the prepared bacterial solution (of a concentration of 5×10⁵ to 5×10⁶ cfu/mL) was added into the first well to the eleventh well, and the twelfth well was not added with the bacterial solution to serve as a blank control. The 96-well plate was placed in a 37° C. incubator, cultured in a static state for 24 hours to observe the bacterial growth in each well. The solution in the well that inhibited bacterial growth was transparent, and the solution in the well that could not inhibit bacterial growth was turbid. The concentration corresponding to the well with a transparent solution was selected to be the minimum inhibitory concentration (MIC) of the sample.

The results are shown in the following tables.

TABLE 1
MIC values of the compounds of the present invention (μg/mL)

| | | Streptococcus | |
|---|---|---|---|
| | Compound | pneumoniae | Escherichia coli |
| Example | No. | ATCC 49169 | ATCC 8099 |
| Example 1 | Ia | 0.391 | 128 |
| Example 2 | Ib | 0.098 | 64 |
| Positive Control | Tylosin | 0.781 | 128 |

As can be seen from Table 1, compared with tylosin, the compound Ia obtained in Example 1 and the compound Ib obtained in Example 2 have a higher in vitro antibacterial activity against Streptococcus pneumoniae (a representative of Gram-positive bacteria), and the compound Ib obtained in Example 2 has a higher in vitro antibacterial activity against Escherichia coli (a representative of Gram-negative bacteria).

TABLE 2
In vitro antibacterial effects of the compound Ia on different strains

| | | MIC (μg/mL) | |
|---|---|---|---|
| | | Before hydrolysis | After hydrolysis |
| Bacteria | No. | of Ia | of Ia |
| Streptococcus | ATCC | 0.391 | 0.391 |
| pneumoniae | 49169 | | |
| Escherichia coli | 8099 | 128 | 128 |
| Staphylococcus | ATCC | 3.125 | 0.781 |
| aureus | 29737 | | |
| Streptococcus | CVCC3940 | 0.391 | 1.563 |
| agalactiae | | | |

As can be seen from Table 2, the antibacterial effects of the compound Ia before and after the hydrolysis thereof on Streptococcus pneumoniae and Escherichia coli are comparable; however, the antibacterial activity against Streptococcus agalactiae is stronger before the hydrolysis of the compound Ia, and the antibacterial activity against Staphylococcus aureus is stronger after the hydrolysis of the compound Ia.

TABLE 3
In vitro antibacterial effects of the compound Ib on different strains

| | | MIC (μg/mL) | |
|---|---|---|---|
| | | Before hydrolysis | After hydrolysis |
| Bacteria | No. | of Ib | of Ib |
| Streptococcus | ATCC | 0.195 | 0.098 |
| pneumoniae | 49169 | | |
| Escherichia coli | ATCC | 64 | 64 |
| | 8099 | | |

As can be seen from Table 3, the antibacterial effects of the compound Ib before and after the hydrolysis thereof on Escherichia coli are comparable; however, the antibacterial activity on Streptococcus pneumoniae is doubled after the hydrolysis of the compound Ib.

Although the present invention has been described in detail above with general descriptions and specific embodiments, it is obvious to those skilled in the art that some modifications or improvements may be made thereto based on the present invention. Therefore, these modifications or improvements made without departing from the spirit of the present invention all fall within the scope claimed by the present invention.

### CROSS-REFERENCE TO RELATED APPLICATIONS

This application claims priority to the Chinese patent application (application number 202311620626.8) filed on Nov. 30, 2023, and the entire contents of that patent application are incorporated herein by reference.

### INDUSTRIAL APPLICATIONS

The present invention has the following technical advantages: the present invention provides a tylosin derivative with a structure represented by in Formula I, which can be used to treat or prevent bacterial infection diseases in animals, and provides more selectivity for treatment with tylosin derivatives.

What is claimed is:

1-16. (canceled)

17. A tylosin derivative, having a structure represented by in Formula Ib:

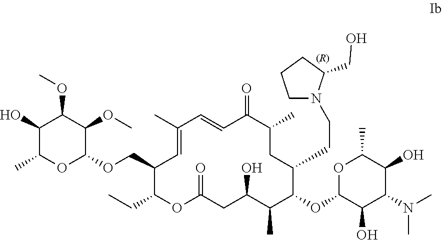

18. A preparation method of the tylosin derivative according to claim 17, comprising the following steps:
S1, subjecting tylosin A and an amino alcohol to a reaction in a non-polar solvent to obtain a reaction solution; wherein the amino alcohol is (R)-prolinol; a molar ratio of the amino alcohol to the tylosin A is from 2:1 to 5:1; and the non-polar solvent is toluene;
S2, adding an acid to the reaction solution, and reacting to obtain a macrolide compound intermediate; wherein the acid is formic acid; the acid is added when a temperature of a reaction system reaches 75-85° C.; a molar ratio of the acid to the tylosin A is from 3:1 to 6:1; and reaction conditions in the step S2 comprise: a temperature of 78-80° C. and a time period of 2-6 h; and
S3, hydrolyzing the macrolide compound intermediate under an acidic condition to obtain the tylosin derivative; wherein an acid is hydrochloric acid; and hydrolyzing conditions comprise: a temperature of room temperature, a time period of 1-6 h, and a concentration of an aqueous solution of the acid of 0.1-10 M.

19. A veterinary pharmaceutical composition, comprising the tylosin derivative according to claim 17.

20. A pharmaceutical preparation, comprising the tylosin derivative according to claim 17.

21. A use of the tylosin derivative according to claim 17, a veterinary pharmaceutical composition comprising the tylosin derivative, or a pharmaceutical preparation comprising the tylosin derivative in preparation of a medicament for treating or preventing a bacterial infection disease in animals;
wherein a bacterium is Streptococcus pneumoniae and/or Escherichia coli.

22. A feed additive, comprising the tylosin derivative according to claim 17.
